(19) United States
(12) Patent Application Publication
(10) Pub. No.: US 2020/0166529 A1
(43) Pub. Date: May 28, 2020

(54) Real-Time Cellular or Pericellular Microenvironmental Oxygen Control

(71) Applicant: AGILENT TECHNOLOGIES, INC., Santa Clara, CA (US)

(72) Inventors: Ian M. Hayes, Glounthaune (IE); James N. Hynes, Ovens (IE)

(21) Appl. No.: 16/611,672

(22) PCT Filed: May 10, 2018
(86) PCT No.: PCT/EP2018/062174

§ 371 Date: Nov. 7, 2019

Related U.S. Application Data
(60) Provisional application No. 62/504,244, filed on May 10, 2017.

(51) Int. Cl. G01N 33/84; G01N 33/50; G01N 33/58

(52) U.S. Cl. CPC G01N 33/84; G01N 33/582; G01N 33/5005

(57) ABSTRACT

A method of maintaining an intracellular oxygen concentration of viable cells forming a cell sample at a target oxygen concentration or maintaining a micro-environmental pericellular space defined by a cell sample of viable cells at a target oxygen concentration, particularly throughout a testing period, by measuring intracellular/pericellular oxygen concentration and adjusting the concentration of oxygen in the surrounding environment in real-time based upon the measured intracellular/pericellular oxygen concentration as necessary and appropriate to influence and maintain the intracellular/pericellular oxygen concentration at a desired target oxygen concentration.

### BACKGROUND

Cell processes, such as signal transduction and gene expression, are highly dependent on the concentration of oxygen that is physiologically available to the cell (i.e., in its immediate proximity) since oxygen is a central requirement for the cell to generate energy. Cellular concentration of oxygen tends to be very different from the concentration of oxygen in the environment (atmosphere or the cell culture media). This is because the cell is constantly consuming oxygen through the energy generating process of mitochondrial aerobic respiration, and rapidly depleting the local concentration of oxygen more rapidly than it can be replaced through diffusion. This creates a gradient of oxygen concentration from the cell (low), through the media (medium) and into the atmospheric phase (high). Furthermore, the concentration of oxygen in tissues of the body can be very different depending upon the location and type of tissue, ranging from 19-20% in cells in the lung (close to atmospheric oxygen), to 5-6% for liver cells, and even to 1-2% in cells deeper in the body (e.g., skeletal muscle).

Even though this difference between atmospheric and cellular oxygen concentrations is known to those who working in specific fields (e.g., hypoxia research), the vast majority of in vitro cell culture experiments conducted across cell biology, including those conducted for disease and drug safety testing, continue to be carried out at 21% (atmospheric) oxygen with no regard paid to the oxygenation levels experienced by the cells under study.

Some researchers, in recognition of the tremendous impact differences in oxygen concentrations can have on test results, attempt to control the concentration of oxygen in the gaseous headspace of test cell cultures by varying and setting the oxygen concentration within the chamber of the cell cultures using specialised equipment—such as an oxygen environment workstation available from Baker Ruskinn of South Wales, England, or the CLARIOstar plate reader equipped with an Atmospheric Control Unit from BMG Labtech GmbH of Ortenberg, Germany.

While use of such equipment to control the concentration of oxygen in the gaseous headspace of test cell cultures, in an effort to more closely mimic in vivo conditions, is a significant advance over the practice of simply performing the tests at 21% (atmospheric) oxygen, a need still exists for an improved system and method of achieving real-time control over the actual pericellular and/or intracellular oxygen concentrations of cells within a cell culture for purposes of more accurately replicating in vivo conditions for oxygen physiologically available to the type of cell under investigation.

### Outline of the Invention

A method of maintaining an intracellular oxygen concentration of viable cells forming a cell sample at a target oxygen concentration or maintaining a micro-environmental pericellular space defined by a cell sample of viable cells at a target oxygen concentration. The method is particularly adapted for maintaining the intracellular or micro-environmental pericellular space oxygen concentration at a target oxygen concentration throughout a test period.

In a first embodiment, the method of the invention comprises the steps of: (A) loading cells in a cell sample with oxygen-sensitive photoluminescent probes to form a loaded cell sample, (B) controlling the concentration of oxygen in environmental fluid communication with the loaded cell sample, (C) ascertaining intracellular oxygen concentration of the loaded cells within the loaded cell sample by (i) detecting an oxygen-sensitive photoluminescent signal emitted by the probes in the loaded cell sample, and (ii) converting the detected oxygen-sensitive photoluminescent signal to a measured intracellular oxygen concentration based upon a known conversion algorithm, (D) comparing the measured intracellular oxygen concentration to a target oxygen concentration, and (E) adjusting the concentration of oxygen in environmental fluid communication with the loaded cell sample in real-time by (i) increasing the concentration of oxygen in environmental fluid communication with the loaded cell sample when the measured intracellular oxygen concentration is below the target oxygen concentration, (ii) decreasing the concentration of oxygen in environmental fluid communication with the loaded cell sample when the measured intracellular oxygen concentration is above the target oxygen concentration, or (iii) maintaining the concentration of oxygen in environmental fluid communication with the loaded cell sample when the measured intracellular oxygen concentration is at the target oxygen concentration.

The step of ascertaining intracellular oxygen concentration of the loaded cells within the loaded cell sample may include at least the steps of (i) exposing the loaded cells in the loaded cell sample to excitation radiation, (ii) measuring radiation emitted by the excited oxygen-sensitive photoluminescent probes loaded within the loaded cells after exposure, and (iii) converting a measured emissions to a measured intracellular oxygen concentration based upon a known conversion algorithm.

In a second embodiment, the method of the invention comprises the steps of: (A) loading cells in a cell sample with oxygen-sensitive photoluminescent probes to form a loaded cell sample, (B) controlling the concentration of oxygen in environmental fluid communication with the loaded cell sample, (C) ascertaining intracellular oxygen concentration of the loaded cells within the loaded cell by: (i) exposing the loaded cells in the loaded cell sample to excitation radiation, (ii) measuring radiation emitted by the excited oxygen-sensitive photoluminescent probes loaded within the loaded cells after exposure, and (iii) converting a measured emissions to a measured intracellular oxygen concentration based upon a known conversion algorithm, (D) comparing the measured intracellular oxygen concentration to a target oxygen concentration, (E) adjusting the concentration of oxygen in environmental fluid communication with the loaded cell sample in real-time by (i) increasing the concentration of oxygen in environmental fluid communication with the loaded cell sample when the measured intracellular oxygen concentration is below the target oxygen concentration, (ii) decreasing the concentration of oxygen in environmental fluid communication with the loaded cell sample when the measured intracellular oxygen concentration is above the target oxygen concentration, or (iii) maintaining the concentration of oxygen in environmental fluid communication with the loaded cell sample when the measured intracellular oxygen concentration is at the target oxygen concentration, and (F) repeating steps (c), (d) and (e) periodically throughout a test period.

The loaded cell sample may be contacted with a drug or drug candidate during the test period.

The steps (c), (d) and (e) may be repeated at least as often as every 20 minutes during the test period. The steps (c), (d) and (e) may be repeated at least as often as every 5 minutes during the test period.

The cells of the method of the invention, or any embodiments thereof, as described herein, may be mammalian cells. The target oxygen concentration of the method of the invention, or any embodiments thereof, as described herein, may be a concentration range.

In the method of the invention, or in any embodiments thereof, as described herein, the loaded cell sample may be formed by (A) incubating the cells in a suitable growth medium containing oxygen-sensitive photoluminescent probes susceptible to cell uptake, (B) washing the incubated cells to remove extracellular probes remaining in the growth medium, and (C) combining the washed cells with a suitable growth medium free of oxygen-sensitive photoluminescent probes.

In the method of the invention, or in any embodiments thereof, as described herein, the oxygen-sensitive photoluminescent probes may be nanoparticulate probes having an average particle size of 20-100 nm.

In the method of the invention, or in any embodiments thereof, as described herein, the amount of any increase or decrease in the concentration of oxygen in environmental fluid communication with the loaded cell sample may be at least 1.5 times the difference between the measured intracellular oxygen concentration and the target oxygen concentration.

In the method of the invention, or in any embodiments thereof, as described herein, the amount of any increase or decrease in the concentration of oxygen in environmental fluid communication with the loaded cell sample may be at least twice the difference between the measured intracellular oxygen concentration and the target oxygen concentration.

In the method of the invention, or in any embodiments thereof, as described herein, the target oxygen concentration may be selected to replicate in vivo intracellular oxygen concentration for the type of cells forming the cell sample.

In a third embodiment, the method of the invention comprises the steps of: (A) loading a pericellular space defined by cells in a cell sample with oxygen-sensitive photoluminescent probes to form a pericellular loaded cell sample, (B) configuring and arranging the pericellular loaded cell sample within an enclosed chamber so as to define a gaseous headspace in fluid communication with the pericellular loaded cell sample within the chamber, and form a micro-environmental pericellular space within the pericellular loaded cell sample, (C) controlling the concentration of oxygen in the gaseous headspace in fluid communication with the pericellular loaded cell sample, (D) ascertaining oxygen concentration within the micro-environmental pericellular space by (i) detecting an oxygen-sensitive photoluminescent signal emitted by the probes in the pericellular loaded cell sample, and (ii) converting the detected oxygen-sensitive photoluminescent signal to a measured micro-environmental pericellular oxygen concentration based upon a known conversion algorithm, (E) comparing the measured micro-environmental pericellular oxygen concentration to a target oxygen concentration, and (F) adjusting the concentration of oxygen in the gaseous headspace in real-time by (i) increasing the concentration of oxygen in the gaseous headspace when the measured micro-environmental pericellular oxygen concentration is below the target oxygen concentration, (ii) decreasing the concentration of oxygen in the gaseous headspace when the measured micro-environmental pericellular oxygen concentration is above the target oxygen concentration, or (iii) maintaining the concentration of oxygen in the gaseous headspace when the measured micro-environmental pericellular oxygen concentration is at the target oxygen concentration.

The step of ascertaining oxygen concentration within a micro-environmental pericellular space may include at least the steps of (i) exposing the pericellular loaded cell sample to excitation radiation, (ii) measuring radiation emitted by the excited oxygen-sensitive photoluminescent probes loaded within the pericellular loaded cell sample, and (iii) converting a measured emissions to a measured pericellular oxygen concentration based upon a known conversion algorithm.

The method may be a method of maintaining a target oxygen concentration within a micro environmental pericellular space defined by a cell sample of viable cells in fluid communication with a surrounding headspace, wherein the micro-environmental pericellular space has an oxygen concentration which differs from the surrounding headspace.

In a fourth embodiment, the method of the invention comprises the steps of: (A) placing oxygen-sensitive photoluminescent probes in sensing fluid communication with a micro-environmental pericellular space defined by a cell sample of viable cells whereby the probes are operable for sensing oxygen concentration within the micro-environmental pericellular space, to form a micro-environmental loaded cell sample, (B) configuring and arranging the micro-environmental loaded cell sample within an enclosed chamber so as to define a gaseous headspace in fluid communication with the micro-environmental loaded cell sample within the chamber, (C) controlling the concentration of oxygen in the gaseous headspace in fluid communication with the micro-environmental pericellular space, (D) ascertaining oxygen concentration within the micro-environmental pericellular space by (i) exposing the micro-environmental loaded cell sample to excitation radiation, (ii) measuring radiation emitted by the excited oxygen-sensitive photoluminescent probes loaded within the micro-environmental loaded cell sample after exposure, and (iii) converting a measured emissions to a measured micro-environmental pericellular space oxygen concentration based upon a known conversion algorithm, (E) comparing the measured micro-environmental pericellular space oxygen concentration to a target oxygen concentration, (F) adjusting the concentration of oxygen in the gaseous headspace in real-time by (i) increasing the concentration of oxygen in the gaseous headspace when the measured micro-environmental pericellular space oxygen concentration is below the target oxygen concentration, (ii) decreasing the concentration of oxygen in the gaseous headspace when the measured micro-environmental pericellular space oxygen concentration is above the target oxygen concentration, or (iii) maintaining the concentration of oxygen in the gaseous headspace when the measured micro-environmental pericellular space oxygen concentration is at the target oxygen concentration, and (G) repeating steps (d), (e) and (f) periodically throughout a test period.

The method may be a maintaining a micro-environmental pericellular space defined by a cell sample of viable cells in fluid communication with a surrounding headspace, at a target oxygen concentration throughout a test period, wherein the micro-environmental pericelluar space has an oxygen concentration which differs from the surrounding headspace.

The pericellular loaded cell sample may be contacted with a drug or drug candidate during the test period.

The steps (d), (e) and (f) may be repeated at least as often as every 20 minutes during the test period. The steps (d), (e) and (f) may be repeated at least as often as every 5 minutes during the test period.

The cells may be mammalian cells.

The target oxygen concentration may be a concentration range.

The amount of any increase or decrease in the concentration of oxygen in the gaseous headspace may be at least 1.5 times the difference between the measured micro-environmental pericellular oxygen concentration and the target oxygen concentration. The amount of any increase or decrease in the concentration of oxygen in the gaseous headspace may be at least twice the difference between the measured micro-environmental pericellular oxygen concentration and the target oxygen concentration.

The target oxygen concentration may be selected to replicate in vivo pericellular oxygen concentration for the type of cells forming the cell sample.

### DETAILED DESCRIPTION OF THE INVENTION

### Maintaining Intracellular Oxygen Concentration

Methods and techniques for intracellular sensing of oxygen by loading cells with suitable oxygen-sensitive photoluminescent probe, reading those probes by detecting an oxygen-sensitive photoluminescent signal emitted by the probes, and converting the detected oxygen-sensitive photoluminescent signals to a measured intracellular oxygen concentration based upon a known conversion algorithm, are widely known as exemplified by WO2012/052068 and US Pat. Appln. Pub 2013/0280751, both incorporated herein by reference. Briefly, cells are commonly loaded with an oxygen-sensitive photoluminescent probe by (A) incubating the cells in a suitable growth medium containing oxygen-sensitive photoluminescent probes susceptible to cell uptake, (B) washing the incubated cells to remove extracellular probes remaining in the growth medium, and (C) combining the washed cells with a suitable growth medium free of oxygen-sensitive photoluminescent probes. Reading of the intracellular probes includes the steps of (i) exposing the loaded cells to excitation radiation, (ii) measuring radiation emitted by the excited oxygen-sensitive photoluminescent probes loaded within the loaded cells after exposure, and (iii) converting a measured emissions to a measured intracellular oxygen concentration based upon a known conversion algorithm. These methods and techniques are suitable for use in providing the intracellular oxygen concentration data necessary to perform the methods of the present invention. The probes are preferably nanoparticulate probes having an average particle size of 20-100 nm. A preferred oxygen-sensitive photoluminescent probe widely recognized for its high loading efficiency, stable luminescent intensity signal and reliable lifetime-based sensing of intracellular oxygen is MitoXpress® Intra, available from Luxcel Biosciences, Ltd of Ireland.

Instruments suitable for reading oxygen-sensitive photoluminescent probes within a cell sample are known and available from a number of sources, including the CLARIOstar plate reader from BMG Labtech GmbH of Ortenberg, Germany.

The intracellular oxygen concentration of viable cells forming a cell sample can be maintained at a target oxygen concentration (or within a target oxygen concentration range) in real-time by using the known methods and techniques referenced supra to measure the intracellular oxygen concentration of the cell sample, and then adjusting the concentration of oxygen in environmental fluid communication with the cell sample in real-time to maintain the target intracellular oxygen concentration by (i) increasing the concentration of oxygen in environmental fluid communication with the cell sample when the measured intracellular oxygen concentration is below the target oxygen concentration, (ii) decreasing the concentration of oxygen in environmental fluid communication with the cell sample when the measured intracellular oxygen concentration is above the target oxygen concentration, or (iii) maintaining the concentration of oxygen in environmental fluid communication with the cell sample when the measured intracellular oxygen concentration is at the target oxygen concentration.

The concentration of oxygen in environmental fluid communication with the loaded cell sample can be achieved by any of several methods known to those of routine skill in the art. One such method is gently flushing the gaseous headspace of the cell culture with replacement gas formed with the desired oxygen concentration. Alternatively, a known quantity of gas containing a known high or low concentration of oxygen can be introduced into the gaseous headspace for increasing or decreasing the concentration of oxygen, respectively. Instruments capable of allowing user control of oxygen and carbon dioxide concentrations in the headspace of a cell sample are known, such as the CLARIOstar plate reader equipped with an Atmospheric Control Unit from BMG Labtech GmbH of Ortenberg, Germany.

These steps can be repeated during and throughout a testing period on a schedule and frequency deemed appropriate, such as every minute, 5 minutes, 10 minutes, 20 minutes, 60 minutes, 120 minutes or indeed any other desired frequency and schedule.

The method is suitable for use in maintaining intracellular oxygen concentration of cell samples formed from a wide variety of viable cells, including specifically but not exclusively 2D and 3D samples of microbial cells, yeast cells and mammalian cells.

Due to an inherent and often significant lag between increases and decreases in the concentration of oxygen in environmental fluid communication with a cell sample, and realization of an increase or decrease in intracellular oxygen concentration effected thereby, it is usually preferred to increase or decrease the concentration of oxygen in environmental fluid communication with a cell sample by at least 1.5 times and more preferably at least twice the difference between the measured intracellular oxygen concentration and the target oxygen concentration.

The target oxygen concentration or concentration range can be selected as desired, but is preferably selected to replicate in vivo intracellular oxygen concentration for the type of cells forming the cell sample, particularly when the cell sample is used for drug testing by contacting the sample with a drug or drug candidate.

### Maintaining Pericellular Oxygen Concentration

Oxygen-sensitive photoluminescent probes capable of sensing and reporting the oxygen concentration of an environment in fluid communication with the probe are widely known. See for example, United States Published Patent Applications 2011/0136247, 2009/0029402, 2008/199360, 2008/190172, 2007/0042412, and 2004/0033575; U.S. Pat. Nos. 8,242,162, 8,158,438, 7,862,770, 7,849,729, 7,749,768, 7,679,745, 7,674,626, 7,569,395, 7,534,615, 7,368,153, 7,138,270, 6,989,246, 6,689,438, 6,395,506, 6,379,969, 6,080,574, 5,885,843, 5,863,460, 5,718,842, 5,595,708, 5,567,598, 5,462,879, 5,407,892, 5,114,676, 5,094,959, 5,030,420, 4,965,087, 4,810,655, and 4,476,870; PCT International Published Application WO 2008/146087; and European Published Patent Application EP 1134583, all of which are hereby incorporated by reference. Such optical sensors are available from a number of suppliers, including Presens Precision Sensing, GmbH of Regensburg, Germany, Oxysense of Dallas, Tex., USA, and Luxcel Biosciences, Ltd of Cork, Ireland.

These probes can be configured, arranged and deployed within a cell culture so that they are concentrated within or in exclusive sensing communication with a pericellular space defined by a sample of viable cells, with the pericellular space forming a micro-environmental having an oxygen concentration which differs from the oxygen concentration within the surrounding headspace of the cell culture. For example, such a pericellular micro-environment can be formed by coating the probes onto the bottom of a culture plate and covering the coating with an adherent mammalian cell type so as to provide a layer of cells separating the probe layer from the gaseous headspace above the cells. Another example is interspersing macro, micro, or nanoparticulate sensors with a mass of viable cells, causing the probe-laden mass to settle to the bottom of the culture, and reading the mass from the bottom of the culture where the probes in the mass are offset from and experience oxygen concentrations within a micro-environment relative to the gaseous headspace above the mass.

Instruments suitable for reading oxygen-sensitive photoluminescent probes within a cell sample are known and available from a number of sources, including the CLARIOstar plate reader from BMG Labtech GmbH of Ortenberg, Germany.

The oxygen concentration within a micro-environmental pericellular space defined by a cell sample of viable cells can be maintained at a target oxygen concentration (or within a target oxygen concentration range) in real-time by using the known methods and techniques referenced supra to measure the pericellular oxygen concentration within the micro-environment, and then adjusting the concentration of oxygen in environmental fluid communication with the cell sample to maintain the target oxygen concentration within the micro-environment by (i) increasing the concentration of oxygen in the gaseous headspace when the measured micro-environmental pericellular oxygen concentration is below the target oxygen concentration, (ii) decreasing the concentration of oxygen in the gaseous headspace when the measured micro-environmental pericellular oxygen concentration is above the target oxygen concentration, or (iii) maintaining the concentration of oxygen in the gaseous headspace the measured micro-environmental pericellular oxygen concentration is at the target oxygen concentration.

The concentration of oxygen in environmental fluid communication with the pericellular loaded cell sample can be achieved by any of several methods known to those of routine skill in the art. One such method is gently flushing the gaseous headspace of the cell culture with replacement gas formed with the desired oxygen concentration. Alternatively, a known quantity of gas containing a known high or low concentration of oxygen can be introduced into the gaseous headspace for increasing or decreasing the concentration of oxygen, respectively. Instruments capable of allowing user control of oxygen and carbon dioxide concentrations in the headspace of a cell sample are known, such as the CLARIOstar plate reader equipped with an Atmospheric Control Unit from BMG Labtech GmbH of Ortenberg, Germany.

These steps can be repeated during and throughout a testing period on a schedule and frequency deemed appropriate, such as every minute, 5 minutes, 10 minutes, 20 minutes, 60 minutes, 120 minutes or indeed any other desired frequency and schedule.

The method is suitable for use in maintaining a target oxygen concentration within a micro-environmental pericellular space defined by a cell sample formed from a wide variety of viable cells, including specifically but not exclusively 2D and 3D samples of microbial cells, yeast cells and mammalian cells.

Due to an inherent and often significant lag between increases and decreases in the concentration of oxygen in environmental fluid communication with a cell sample, and realization of an increase or decrease in oxygen concentration within a micro-environmental pericellular space defined by a cell sample of viable cells effected thereby, it is usually preferred to increase or decrease the concentration of oxygen in environmental fluid communication with a cell sample by at least 1.5 times and more preferably at least twice the difference between the measured oxygen concentration within a micro-environmental pericellular space defined by a cell sample of viable cells and the target oxygen concentration.

The target oxygen concentration or concentration range can be selected as desired, but is preferably selected to replicate in vivo pericellular oxygen concentration for the type of cells forming the cell sample, particularly when the cell sample is used for drug testing by contacting the sample with a drug or drug candidate.

The invention is not limited to the embodiments hereinbefore described which may be varied in construction and detail without departing from the invention. It will be appreciated that embodiments or preferred features thereof as described herein may be applied to any method of the invention, or embodiment thereof.

1. A method of maintaining an intracellular oxygen concentration of viable cells forming a cell sample at a target oxygen concentration, comprising the steps of:
(a) loading cells in a cell sample with oxygen-sensitive photoluminescent probes to form a loaded cell sample,
(b) controlling the concentration of oxygen in environmental fluid communication with the loaded cell sample,
(c) ascertaining intracellular oxygen concentration of the loaded cells within the loaded cell sample by (i) detecting an oxygen-sensitive photoluminescent signal emitted by the probes in the loaded cell sample, and (ii) converting the detected oxygen-sensitive photoluminescent signal to a measured intracellular oxygen concentration based upon a known conversion algorithm,
(d) comparing the measured intracellular oxygen concentration to a target oxygen concentration, and
(e) adjusting the concentration of oxygen in environmental fluid communication with the loaded cell sample in real-time by (i) increasing the concentration of oxygen in environmental fluid communication with the loaded cell sample when the measured intracellular oxygen concentration is below the target oxygen concentration, (ii) decreasing the concentration of oxygen in environmental fluid communication with the loaded cell sample when the measured intracellular oxygen concentration is above the target oxygen concentration, or (iii) maintaining the concentration of oxygen in environmental fluid communication with the loaded cell sample when the measured intracellular oxygen concentration is at the target oxygen concentration.

2. The method of claim 1 wherein the step of ascertaining intracellular oxygen concentration of the loaded cells within the loaded cell sample includes at least the steps of (i) exposing the loaded cells in the loaded cell sample to excitation radiation, (ii) measuring radiation emitted by the excited oxygen-sensitive photoluminescent probes loaded within the loaded cells after exposure, and (iii) converting a measured emissions to a measured intracellular oxygen concentration based upon a known conversion algorithm.

3. A method of maintaining an intracellular oxygen concentration of viable cells forming a cell sample at a target oxygen concentration throughout a test period, comprising the steps of:
(a) loading cells in a cell sample with oxygen-sensitive photoluminescent probes to form a loaded cell sample,
(b) controlling the concentration of oxygen in environmental fluid communication with the loaded cell sample,
(c) ascertaining intracellular oxygen concentration of the loaded cells within the loaded cell by: (i) exposing the loaded cells in the loaded cell sample to excitation radiation, (ii) measuring radiation emitted by the excited oxygen-sensitive photoluminescent probes loaded within the loaded cells after exposure, and (iii) converting a measured emissions to a measured intracellular oxygen concentration based upon a known conversion algorithm,
(d) comparing the measured intracellular oxygen concentration to a target oxygen concentration,
(e) adjusting the concentration of oxygen in environmental fluid communication with the loaded cell sample in real-time by (i) increasing the concentration of oxygen in environmental fluid communication with the loaded cell sample when the measured intracellular oxygen concentration is below the target oxygen concentration, (ii) decreasing the concentration of oxygen in environmental fluid communication with the loaded cell sample when the measured intracellular oxygen concentration is above the target oxygen concentration, or (iii) maintaining the concentration of oxygen in environmental fluid communication with the loaded cell sample when the measured intracellular oxygen concentration is at the target oxygen concentration, and
(f) repeating steps (c), (d) and (e) periodically throughout a test period.

4. The method of claim 3 wherein the loaded cell sample is contacted with a drug or drug candidate during the test period.

5. The method according to claim 3 wherein steps (c), (d) and (e) are repeated at least as often as every 20 minutes during the test period.

6. (canceled)

7. The method according to claim 1 wherein the cells are mammalian cells.

8. The method according to claim 1 wherein the target oxygen concentration is a concentration range.

9. The method according to claim 1 wherein the loaded cell sample is formed by (A) incubating the cells in a suitable growth medium containing oxygen-sensitive photoluminescent probes susceptible to cell uptake, (B) washing the incubated cells to remove extracellular probes remaining in the growth medium, and (C) combining the washed cells with a suitable growth medium free of oxygen-sensitive photoluminescent probes.

10. The method according to claim 1 wherein the oxygen-sensitive photoluminescent probes are nanoparticulate probes having an average particle size of 20-100 nm.

11. The method according to claim 1 wherein the amount of any increase or decrease in the concentration of oxygen in environmental fluid communication with the loaded cell sample is at least 1.5 times the difference between the measured intracellular oxygen concentration and the target oxygen concentration.

12. The method according to claim 3 wherein the amount of any increase or decrease in the concentration of oxygen in environmental fluid communication with the loaded cell sample is at least twice the difference between the measured intracellular oxygen concentration and the target oxygen concentration.

13. The method according to claim 1 wherein the target oxygen concentration is selected to replicate in vivo intracellular oxygen concentration for the type of cells forming the cell sample.

14. A method of maintaining a target oxygen concentration within a micro-environmental pericellular space defined by a cell sample of viable cells in fluid communication with a surrounding headspace, wherein the micro-environmental pericellular space has an oxygen concentration which differs from the surrounding headspace, the method comprising the steps of:
(a) loading the pericellular space defined by cells in a cell sample with oxygen-sensitive photoluminescent probes to form a pericellular loaded cell sample,
(b) configuring and arranging the pericellular loaded cell sample within an enclosed chamber so as to define a gaseous headspace in fluid communication with the pericellular loaded cell sample within the chamber, and form a micro-environmental pericellular space within the pericellular loaded cell sample,
(c) controlling the concentration of oxygen in the gaseous headspace in fluid communication with the pericellular loaded cell sample,
(d) ascertaining oxygen concentration within the micro-environmental pericellular space by (i) detecting an oxygen-sensitive photoluminescent signal emitted by the probes in the pericellular loaded cell sample, and (ii) converting the detected oxygen-sensitive photoluminescent signal to a measured micro-environmental pericellular oxygen concentration based upon a known conversion algorithm,
(e) comparing the measured micro-environmental pericellular oxygen concentration to a target oxygen concentration, and
(f) adjusting the concentration of oxygen in the gaseous headspace in real-time by (i) increasing the concentration of oxygen in the gaseous headspace when the measured micro-environmental pericellular oxygen concentration is below the target oxygen concentration, (ii) decreasing the concentration of oxygen in the gaseous headspace when the measured micro-environmental pericellular oxygen concentration is above the target oxygen concentration, or (iii) maintaining the concentration of oxygen in the gaseous headspace when the measured micro-environmental pericellular oxygen concentration is at the target oxygen concentration.

15. The method of claim 14 wherein the step of ascertaining oxygen concentration within a micro-environmental pericellular space includes at least the steps of (i) exposing the pericellular loaded cell sample to excitation radiation, (ii) measuring radiation emitted by the excited oxygen-sensitive photoluminescent probes loaded within the pericellular loaded cell sample, and (iii) converting a measured emissions to a measured pericellular oxygen concentration based upon a known conversion algorithm.

16. A method of maintaining a micro-environmental pericellular space defined by a cell sample of viable cells in fluid communication with a surrounding headspace, at a target oxygen concentration throughout a test period, wherein the micro-environmental pericelluar space has an oxygen concentration which differs from the surrounding headspace, the method comprising the steps of:
(a) placing oxygen-sensitive photoluminescent probes in sensing fluid communication with the micro-environmental pericellular space defined by a cell sample of viable cells whereby the probes are operable for sensing oxygen concentration within the micro-environmental pericellular space, to form a micro-environmental loaded cell sample,
(b) configuring and arranging the micro-environmental loaded cell sample within an enclosed chamber so as to define a gaseous headspace in fluid communication with the micro-environmental loaded cell sample within the chamber,
(c) controlling the concentration of oxygen in the gaseous headspace in fluid communication with the micro-environmental pericellular space,
(d) ascertaining oxygen concentration within the micro-environmental pericellular space by (i) exposing the micro-environmental loaded cell sample to excitation radiation, (ii) measuring radiation emitted by the excited oxygen-sensitive photoluminescent probes loaded within the micro-environmental loaded cell sample after exposure, and (iii) converting a measured emissions to a measured micro-environmental pericellular space oxygen concentration based upon a known conversion algorithm,
(e) comparing the measured micro-environmental pericellular space oxygen concentration to a target oxygen concentration,
(f) adjusting the concentration of oxygen in the gaseous headspace in real-time by (i) increasing the concentration of oxygen in the gaseous headspace when the measured micro-environmental pericellular space oxygen concentration is below the target oxygen concentration, (ii) decreasing the concentration of oxygen in the gaseous headspace when the measured micro-environmental pericellular space oxygen concentration is above the target oxygen concentration, or (iii) maintaining the concentration of oxygen in the gaseous headspace when the measured micro-environmental pericellular space oxygen concentration is at the target oxygen concentration, and
(g) repeating steps (d), (e) and (f) periodically throughout a test period.

17. The method of claim 16 wherein the pericellular loaded cell sample is contacted with a drug or drug candidate during the test period.

18. The method according to claim 16 wherein steps (d), (e) and (f) are repeated at least as often as every 20 minutes during the test period.

19. (canceled)

20. (canceled)

21. The method according to claim 14 wherein the target oxygen concentration is a concentration range.

22. (canceled)

23. The method according to claim 14 wherein the amount of any increase or decrease in the concentration of oxygen in the gaseous headspace is at least twice the difference between the measured micro-environmental pericellular oxygen concentration and the target oxygen concentration.

24. The method according to claim 14 wherein the target oxygen concentration is selected to replicate in vivo pericellular oxygen concentration for the type of cells forming the cell sample.
